(19) United States
(12) Patent Application Publication
(10) Pub. No.: US 2011/0053211 A1
(43) Pub. Date: Mar. 3, 2011

(54) PROCESS AND DEVICE FOR DETERMINING THE CONDITION OF BIOLOGICAL MATERIAL, IN PARTICULAR FOOD

(76) Inventor: Bernard Douet, Villars-Sainte-Croix (FR)

(21) Appl. No.: 12/160,788

(22) PCT Filed: Jan. 13, 2006
(86) PCT No.: PCT/AT06/00014

§ 371 Date: Sep. 19, 2008

(51) Int. Cl. G01N 21/55; C12Q 1/04; C12M 1/34

(52) U.S. Cl. 435/34; 356/448; 435/288.7

(57) ABSTRACT

The invention relates to a process for determining—remotely and without taking samples—the condition of biological material, in particular food, as well as a device for performing this process. Radiation emission is thus induced with coherent beams in the material to be examined and is directly measured, whereby the measured values are compared to a nominal or boundary value. To this end, the device has a radiation source for emitting coherent beams, a detector for determining the induced radiation emission, and a control device, whereby the control device contains a microcomputer unit for comparison of the determined radiation emission with the nominal and boundary values that are stored in the memory.

This invention relates to a process for determining—remotely and without taking samples—the condition of biological material, in particular food, as well as a device for performing this process.

It is known that chemical conversions of substances that cause biological changes in materials can be carried out by microbiological processes. This can cause food to spoil, etc.

In connection with the problem of this checking for spoilage of foods, various processes are known. In CH 564 775, a process is disclosed in which the packaging to be examined is stored under uniform thermal conditions in a storage space. This must be carried out under a specific temperature and over a period of a few hours or days, after which the assessment is then carried out.

AT 324,026 describes an expensive process in which the examination is performed in a laboratory with the aid of a thermal imaging camera, whereby in this process, not a composition of the examined material but rather only an elevated bacteria content is determined.

The process that is described in AT 384,679 deals with the detection of yeast and mold, whereby in this process, the material that is to be examined in a laboratory is subjected to a cultivation process, which also claims a high expenditure in time, material and personnel.

In EP 0 311 177, the examination is performed with the aid of elastic, scattered radiation, whereby this examination is determined with highly technical devices for checking pieces of luggage or the like for undesirable contents.

Also, measuring processes with x-rays are already known from U.S. Pat. No. 3,973,128. Also, the media have already reported a device for detecting hazardous bacteria, in which, however, the procedure is performed with the refraction intensity and diffraction angles.

Until now, laser measurement technology was used only for detecting certain substances, for example to conduct checks at border-crossing points, for detecting banned and dangerous materials (drugs or explosive materials and weapons). That is to say that known measuring techniques (by measuring the emission of beams, such as, e.g., laser, atomic laser, alpha-spectrometer measurements) relate only to checking for the presence of a certain specifically known material, but not the detection of its biological condition or optionally its biological composition.

All processes for checking the condition of a biological material that are known from the prior art require an expensive examination process. With the aid of technically highly-qualified personnel, complicated devices, and well-equipped workstations, many process steps were often necessary, whereby the measuring result often is applicable only to a certain product (e.g., milk).

Furthermore, the factors of time, temperature and environment should not be disregarded in studies that have to be performed not on site but rather only in a laboratory.

Materials to be examined can be further changed biologically by the above-mentioned circumstances during transport into a laboratory and then no longer correspond to the material that was removed.

The number of diseases that increases steadily by the consumption of spoiling materials shows the necessity of developing a quick, simple testing process, which also makes it possible for a layman to perform an exact quality control. In most cases, a large number of people are often affected immediately, for example in the case of eating facilities in schools, after-school day care, play groups or nursing homes.

By certificates of origin and the like, many firms seek to provide a certain safety to the consumers when they purchase their goods; however, the best goods can spoil prematurely during shipping and if improperly stored.

It is therefore certainly in accordance with the firm to offer to the consumer safety when purchasing perishable foods, which can be performed quickly, economically and on site, whereby also a connection to the known reading devices on cash registers is possible.

From DE 27 28 717 A1, a determination—remotely and without taking samples—is known. In this known design, the surfaces of the test objects to be examined are scanned, e.g., also with a laser light, whereby the reflected or emitted radiation is measured. Thus, virtually only the surface of the test object, but not the lower-lying layers, is examined.

According to the invention, the above-mentioned drawbacks are avoided, in that in the material to be examined, radiation emission is induced by means of coherent beams and is measured directly, whereby the measured values are compared to a nominal or boundary value. Thus, even layers located below the surface are examined, by which the overall condition of the material to be examined can be detected. In this case, the process according to the invention makes use of the circumstance that any change in the biological composition or the biological structure of a material, or a microorganism attack induces a change in the induced radiation emission. Because of the known change and the comparison to a nominal or boundary value, it can then be determined in a simple way whether the examined material corresponds to the specified standards or not.

Advantageously, the nominal values (optimum condition) or the boundary values for the radiation emission can be determined as guide values and then stored. This is carried out best of all in that various conditions that can occur in practice are simulated artificially in a laboratory, e.g., contamination or infection of foods with microorganisms, pathogens, viruses or other pathogens. The deviations that occur relative to the already measured and stored nominal or boundary value are then used to evaluate the condition of the material that is examined. The determined data can be stored together in principle just with time and date information for comparison measurements in an advantageous way.

In this case, the initial output of the emitted radiation can be slightly below the acceptable output limit.

This acceptable and/or legally permitted output limit of the laser ensures that even when used by experts, other individuals who are in the vicinity of the measuring device are not injured. In addition, there is no danger that, in the case of excessive power, the examined food will distort the initial output of the emitted beams with consideration of the packaging. In the same manner, the initial output of the emitted radiation can be matched to the removal between the radiation source and the material to be examined, by which a more exact measurement is made possible.

In a device according to the invention for performing the process according to the invention, which has a radiation source for emitting coherent beams to a detector for determining the induced radiation emission and a control device, the beam device contains a microcomputer unit for comparing the determined emission data to the nominal and boundary values stored in the memory.

In an advantageous way, the device can be specialized by expansion (add-on chips, add-on cards) even for detecting special microbiological processes (e.g., when traveling).

In particular in this connection, it can be suitable for detecting bacteria, yeast and mold, whereby optionally also simultaneously one after another, characteristic properties such as germ content, gas formation, fermentation activity, acid formation and the like can be determined.

For certain applications, special parameters can be stored in memory, by which detailed examinations can be made for defined bacteria, e.g., salmonella in food, mold in milk, or specific pathogens in liquids. Furthermore, the device can be adjusted such that it offers a control of the spraying agent and/or fertilizer that are used with fruit and vegetables and/or addition of possibly banned preservatives. In this case, the radiation can be controlled such that by the control of the emitted radiation, food can be examined for its suitability for consumption independently of the temperature, i.e., even frozen food.

In this case, the memory can be provided with data that in addition make possible an examination of the composition and/or strength of the packaging materials and/or the release of certain substances. Finally, the device can be connected to reading processes, known in the art, at cash registers, whereby the result can be brought up on the register receipt. In the drawing, diagrammatic structures are shown, specifically in

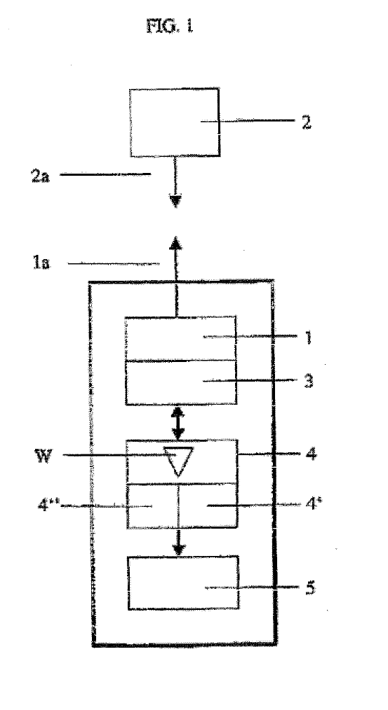

FIG. 1 the design of a basic device, in

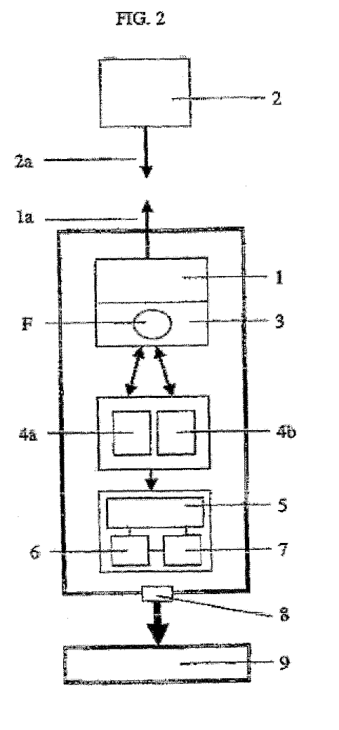

FIG. 2 a preferred embodiment, with which a precise diagnosis of the content of the material to be examined is made possible, and

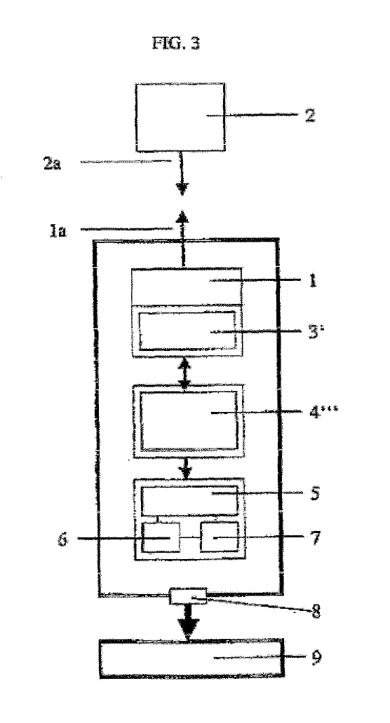

FIG. 3 another variant embodiment of the device according to the invention.

In this case, according to FIG. 1, the laser beam 1a, which is emitted by laser 1, strikes the material 2 to be examined and induces an emission beam 2a, which is stored by a central microcomputer unit 3. Preferably, a switch W, which is referred to below as a selector switch for short, is incorporated in the device. By the latter, a switching to the subgroups 4′ and 4″ stored in memory 4, which contain the nominal values—in this case, these are defined boundary values of the desired introductory clauses, e.g., milk or meat, i.a., whereby these are at least two subgroups—is made possible.

A comparison of the measuring result with the stored nominal value is carried out on the central microcomputer unit 3. The result that is determined is shown in analog or digital form on the display device 5, and it can be supported by acoustic and/or optical signals, and the result is limited only to a positive or negative result, e.g., the milk is suitable for consumption—yes/no, without a definition of the content.

FIG. 2 shows the diagrammatic design of another preferred embodiment of the device according to the invention, whereby, as indicated, a more precisely worded diagnosis of the content of the material to be examined is made possible. This is achieved by comparison of the induced radiation emission 2a on the central microcomputer unit 3 with stored nominal and boundary values of the independent memories, whereby, for example, one of the memories is used for the area of food and the other is used for medical purposes, as already mentioned above in the description (referred to below as reference memories 4a and 4b for short), whereby it can be switched by a functional switch F between at least two reference memories 4a and 4b. The result that is determined is mentioned in the description, and is used (referred to below as reference memories 4a and 4b for short), whereby it can be switched by a functional switch F between at least two reference memories 4a and 4b. The result that is determined is shown in analog or digital form in the display device 5, whereby it can be supported by acoustic and/or optical signals. There also exists the possibility of supplementing the result with date and time information 6 and/or storing by means of a memory chip 7 and/or printing out the result via an interface 8 by means of a writing device 9.

FIG. 3 clarifies the diagrammatic design of another form of the device according to the invention, whereby the central microcomputer unit 3′ is supplemented with a multi-data storage unit for serial diagnostics or analysis, which makes possible, on the one hand, the detection of, e.g., salmonella and, on the other hand, a medical analysis, in which as much information of the induced emission beam 2a as possible is stored as an actual value and is compared to as many nominal values as possible from the reference memory 4″′, by which the possibility of the actual detection of certain bacteria, yeast and/or mold, salmonella, i.a., is made possible. Also, here, the result that is determined, as already described in FIG. 2, is shown in analog or digital form on the display device 5, whereby it can be supported by acoustic and/or optical signals. There also exists the possibility of supplementing the result with date and time information 6 and/or storing by means of a memory chip 7 and/or printing out the result via an interface 8 by means of a writing device 9.

In FIGS. 2 and 3, components that remain the same are not described again; they are visible from the drawings and have been referred to with reference numbers that remain the same in the preceding figure. Since the devices according to the invention, primarily in the acceptable variants, are miniature devices with low power consumption, the power supply can be easily produced by means of conventional batteries, e.g., long-term batteries, but a direct power supply by means of a mains adapter is also possible. The elements that are necessary for this purpose are not shown in FIGS. 1 to 3.

The best comparison to the various production stages of the device according to the invention are the pocket calculators known in the art, which range from the simple and economical model with basic calculation features to the sophisticated pocket calculators with advanced technical functions. Other embodiments that are not cited individually here can be easily derived for one skilled in the art who is familiar with this field. The feature of the invention consists in that for the first time, a diagnostic process is made possible independently of the packaging.

In this case, the material to be examined no longer has to be removed from the packaging, whereby also the type of packaging (plastic, glass, metal, or composite materials such as Tetra-Pack, or in a vacuum, i.a.) does not affect the result. It must only be taken into account that the level of the emitted laser beam of the device according to the invention is selected such that an emission is made possible. To obtain the measuring result, care must be taken to ensure that a higher energy level is also more fully occupied. Then, the rate of induced emission is higher owing to light radiation than the adsorption of the light radiation; it is not weakened, but rather intensified during passage through the material.

To obtain the most reliable values possible, the ratio between the minimum and maximum distances from the material to be measured and the intensity of the emission beam (of the measuring beam emitted from the device) must be taken into consideration. As already known, laser technology is already used in medicine, i.a., also in eye examinations or treatments. In the process according to the invention or the device according to the invention, attention is to be paid to the fact that the maximum initial output radiated by the device according to the invention (energy intensity of the emitted beam) is just under the acceptable output limit. As a result, it is provided that even with unintentional tampering with the device produced according to the invention, the health risk is minimized. A corresponding warning notice on the device to warn, for example, children, i.a., against careless handling is recommended, however.

An especially economical embodiment of the device provides that the device according to the invention can examine the material to be examined for specific bacteria. In this case, the device must be characterized by its special readiness for use. According to an especially preferred embodiment of the process according to the invention, detection both of bacteria, yeast and mold is possible; also simultaneously one after the other, characteristic properties such as germ content, gas formation, fermentation activity, acid formation, etc., can be determined. This is especially advantageous in detecting salmonella or fungus poisoning, since in these cases, the symptoms occur in salmonella after 8 to 14 hours and in fungus poisoning after up to 18 hours.

Mycotoxins, which are formed by mold, harbor a very widespread problem. They can be present in all foods, are odorless and tasteless, and can result in severe liver damage. Up until now, the detection of mycotoxins was difficult and time-consuming. Aflatoxins, which occur in, for example, pistachios, are a known subgroup of mycotoxins.

With the device that is produced according to the invention, the suitability for consumption of liquids, e.g., milk, in known packaging (for example, Tetra-pack) can be examined in an especially simple way. In this case, the device is supported on studies in which sealed, aseptic packages are stored under uniform thermal conditions in a storage space at temperatures of between +2° and +60°, preferably about +15° C. to +25° C., over several hours or days. The thus determined results can be stored in the device as boundary values.

It can also be determined by the determination of certain products that arise during spoilage, e.g., of adenosine triophosphate, such as hypoxanthine, inosine and inosinic acid and/or the compounding thereof depending on the exposure sensitivity by means of specific enzymes, i.e.,

Hypoxanthine-xanthine-oxidase,

Inosine-nucleoside phosphorylase,

Inosinic acid-alkaline phosphatase nucleoside phosphorylase and xanthine-oxidase=xanthine (C5H4N4O2).

The device that is produced according to the invention makes possible the detection of the spraying agent or fertilizer that is used with fruit and vegetables and/or the existence and/or addition of possibly banned preservatives even in foods in processed form, e.g., in jelly or ketchup.

Furthermore, the process according to the invention or the device according to the invention offers the possibility to examine food independently of the temperature, i.e., even frozen foods, for the suitability for consumption thereof. One almost unnoticed fact consists in that microorganisms are not killed by cooling. Many enzymes also operate at temperatures of −40° C. Frozen food, which recently has become more and more common on grocery shelves, is especially susceptible because of the thorny problem of shipping (danger of breaking the cold chain). The device according to the invention can be manufactured so that it is specialized by expansions (add-on chips, add-on cards) even for detecting special microbiological processes or for detecting microorganisms. This is primarily very effective when traveling, in which by other conditions of hygiene or by high temperatures, it results in quick spoiling of the food. Also, checking the composition and/or strength of the packaging materials and/or the release of certain substances (e.g., release of cancer-producing parts from many packaging materials) in food is possible. In order to offer to the consumer as important information as possible on the packaged or unpackaged liquid, solid, raw or processed food, as indicated above, it is also possible to combine the device according to the invention with the reading processes, known in the art, at cash registers. In food in which suitability for consumption is diminished, the result that is determined can be printed out on the register receipt.

Furthermore, the device according to the invention or the process according to the invention can be used especially efficiently in research for determining the condition of biological material, which essentially is characterized in that the induced radiation emission, emitted from the material to be examined, irradiated with a radiation source, e.g., laser, is measured directly, so that it is compared to at least one nominal or boundary value, by which it was possible to shorten time-intensive examination processes, and to accelerate, e.g., the development of vaccines, i.a. With the process according to the invention or the device according to the invention, a determination and diagnosis of all biological materials is therefore possible in an especially simple way. Since even blood and other bodily substances consist of biological materials, in which microbiological changes take place, it is logical to also detect here these microbiological changes with the process according to the invention or the device according to the invention. Another advantageous embodiment of the invention consists in that it makes it possible to discover microbiological changes, which take place in other organic substances, such as, e.g., blood, i.a., with a single device by means of control switches, since, for example, each germ releases a characteristic induced emission beam, whereby the examination can be performed on the site and without physical contact with the material to be studied (i.e., to be taken without blood, i.a.), and by any individual without technical knowledge at low expense on site. Inflammations or other organic changes, such as, for example, cancer, can thus be determined quickly and simply.

In an especially simple way, an elevated glucose level, which releases another induced radiation emission, can be detected in the blood (hyperglycemia), which is the most important clinical sign for detecting diabetes (diabetes mellitus). To date, regular blood sugar determinations were performed in patients with the aid of test rods.

Another characteristic feature of this metabolic disease is the excretion of glucose in urine.

Healthy patients thus indicate a fasting glucose of

Capillary blood 55-100 mg/dl

Venous blood 55-100 mg/dl.

In patients with diabetes, the following values occur one hour after eating:

In the capillary blood over 200 mg/dl

In the venous blood over 180 mg/dl.

Also, these already known values can be used as nominal or actual values. Also, even in the uric acid, measured values (males: 3.5-7.1 mg/dl and females: 2.5-5.9 mg/dl) are known. Urobilinogens (upper limits of normal uribilinogen excretion 1 mg/100 ml) are increased in patients with acute and chronic liver inflammation or are indicated for detecting toxic liver damage or liver tumors. Also here, the invention now introduces its object that consists in making possible a simple and—for the patients—as painfree a diagnosis as possible without required removal of the organic substance that is to be examined. It is also conceivable to introduce the device according to the invention for determining the blood group. This is especially advantageous if a quick diagnosis is necessary after accidents or emergency operations.

A preferred and simple variant embodiment of the invention provides for the possibility of detecting narcotics and drugs, even in the chemically produced forms of narcotics and drugs, in the body, e.g., in the blood.

The possibility also exists, depending on the legal determinations in the country of manufacture, to produce a combination of the device with an automobile starter to make possible—before starting up—a checking of the driving capability of the drive rod and optionally to prevent the motor vehicle from being started.

Furthermore, a combination of the device with cameras, mobile telephones, clocks, etc., is also possible, which considerably facilitates the preservation of evidence in case of doubt, whereby in addition, time and date information can be stored. The device that is produced according to the invention can show the determined measured values in an analog or digitally readable form and/or can be equipped with acoustic and/or optically perceivable signals.

1. Process for determining—remotely and without taking samples—the condition of biological material, in particular food, characterized in that radiation emission is induced with coherent beams in the material to be examined and directly measured, whereby the measured values are compared to a nominal or boundary value.

2. Process according to claim 1, wherein as guide values, the nominal value (optimum condition) or boundary values are determined for the radiation emission and then stored.

3. Process according to claim 2, wherein additional time and date information for comparison measurements are stored together.

4. Process according to claim 1, wherein the initial output of the emitted radiation can be slightly below the acceptable output limit.

5. Process according to claim 4, wherein the initial output of the emitted radiation is selected with consideration of the packaging, in particular the type of packaging material.

6. Process according to claim 4, wherein the initial output of the emitted radiation is matched to the removal between the radiation source and the material to be examined.

7. Process according to claim 1, wherein the material to be examined is tissue, blood or urine, in particular from humans.

8. Process according to claim 7, wherein the material to be examined is determined on site without removal and physical contact.

9. Device for performing the process according to claim 1, with a radiation source for emitting a coherent beam, a detector for emitting the induced radiation emission and a control device, wherein the control device contains a microcomputer unit (3) for comparison of the emitted radiation emission with the nominal value (4′) and boundary value (4″) that are stored in memory (4).

10. Device according to claim 9, wherein it is specialized by expansions (add-on chips, add-on cards) even for detecting special microbiological processes (e.g., when traveling).

11. Device according to claim 9, wherein it is suitable for detecting both bacteria, yeast and mold, whereby optionally also simultaneously one after the other, characteristic properties, such as germ content, gas formation, fermentation activity, acid formation, etc., are determined.

12. Device according to claim 9, wherein special parameters are stored in memory (4), by which e.g., salmonella in food, mold in milk, or specific pathogens in liquids can be examined, presented in detail according to defined bacteria.

13. Device according to claim 9, wherein it offers a control of the spraying agent and/or fertilizer that is used with fruit and vegetables and/or addition of possibly banned preservatives.

14. Device according to claim 9, wherein by adjusting the emitted radiation, the food, independently of the temperature, i.e., also frozen food, can be examined for its suitability for consumption.

15. Device according to claim 9, wherein the memory (4) is provided with data, which in addition make possible an examination of the composition and/or strength of packaging materials and/or the release of specific substances.

16. Device according to claim 9, wherein it is connected to reading processes, known in the art, at cash registers, whereby the result can be brought up on the register receipt.

17. Process according to claim 2, wherein the initial output of the emitted radiation can be slightly below the acceptable output limit.

18. Process according to claim 5, wherein the initial output of the emitted radiation is matched to the removal between the radiation source and the material to be examined.

19. Process according to claim 2, wherein the material to be examined is tissue, blood or urine, in particular from humans.

20. Device according to claim 10, wherein it is suitable for detecting both bacteria, yeast and mold, whereby optionally also simultaneously one after the other, characteristic properties, such as germ content, gas formation, fermentation activity, acid formation, etc., are determined.
